(19) United States
(12) Patent Application Publication
(10) Pub. No.: US 2008/0280768 A1
(43) Pub. Date: Nov. 13, 2008

(54) Reagents and Methods for Predicting Drug Resistance

(75) Inventors: William A. Ricketts, Irvine, CA (US); David L. Smith, Santa Ana, CA (US); Snjezana Covic, Cypress, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Oncotech, Inc., Tustin, CA (US)

(21) Appl. No.: 12/023,021

(22) Filed: Jan. 30, 2008

Related U.S. Application Data
(60) Provisional application No. 60/887,269, filed on Jan. 30, 2007.

(51) Int. Cl. C12Q 1/68; C40B 20/00; G01N 33/00; C07H 21/00; G01N 33/50

(52) U.S. Cl. 506/2; 435/6; 435/7.1; 435/7.92; 536/24.31

(57) ABSTRACT

The invention provides methods for prognosis, diagnosis, staging and determining disease progression in human cancer patients related to amplification levels of one or a plurality of genetic loci that are differentially amplified in chemotherapeutic drug resistant tumor cells.

This application claims priority to U.S. Provisional Application No. 60/887,269 filed Jan. 30, 2007, which is hereby incorporated by reference herein in its entirety, including the drawings.

### BACKGROUND OF THE INVENTION

1. Field of the Invention

The invention relates to prognostic methods for determining an effective course of chemotherapy for a cancer patient, more specifically by early detection of a drug resistance phenotype in neoplastic cells obtained from cancer patients, before or during the course of chemotherapeutic treatment. The invention in particular relates to the identification of amplified regions within tumor cell genomic DNA specific for tumors that are resistant to particular chemotherapeutic agents or classes of chemotherapeutic agents. The methods of the invention are specifically directed to breast cancer, colorectal cancer, ovarian cancer, and non-small cell lung cancer cells, but the generality of the method is directed to cancer cells of any cell or tissue of origin. In the practice of the methods of the invention are used and provided by the invention a plurality of genetic loci that are genetically amplified and/or exhibit increased expression in drug resistant neoplastic cells. The invention provides methods for identifying such genetic loci that are amplified as well as methods for using this information to make clinical decisions on cancer treatment, especially chemotherapeutic drug treatment of cancer patients.

2. Summary of the Related Art

Cancer remains one of the leading causes of death in the United States. Clinically, a broad variety of medical approaches, including surgery, radiation therapy and chemotherapeutic drug therapy are currently being used in the treatment of human cancer (see the textbook CANCER: Principles & Practice of Oncology, 7th Edition, De Vita et al., eds., Lippincott Williams and Wilkins, Philadelphia, Pa., 2005). However, it is recognized that such approaches continue to be limited by a fundamental inability to accurately predict the likelihood of clinically successful outcomes, particularly with regard to the sensitivity or resistance of a particular patient's tumor to a chemotherapeutic agent or combinations of chemotherapeutic agents.

A broad variety of chemotherapeutic agents are used in the treatment of human cancer. These include the plant alkaloids vincristine, vinblastine, vindesine, and VM-26; the antibiotics actinomycin-D, doxorubicin, daunorubicin, mithramycin, mitomycin C and bleomycin; the antimetabolites methotrexate, 5-fluorouracil, 5-fluorodeoxyuridine, 6-mercaptopurine, 6-thioguanine, cytosine arabinoside, 5-aza-cytidine and hydroxyurea; the alkylating agents cyclophosphamide, melphalan, busulfan, CCNU, MeCCNU, BCNU, streptozotocin, chlorambucil, bis-diamminedichloroplatinum, azetidinylbenzoquinone; and the miscellaneous agents dacarbazine, mAMSA and mitoxantrone (DeVita et al., Id.). However, some neoplastic cells become resistant to specific chemotherapeutic agents, in some instances even to multiple chemotherapeutic agents, and some tumors are intrinsically resistant to certain chemotherapeutic agents. Such drug resistance or multiple drug resistance can theoretically arise from expression of genes that confer resistance to the agent, or from lack of expression of genes that make the cells sensitive to a particular anticancer drug. One example of the former type is the multidrug resistance gene, MDR1, which encodes an integral plasma membrane protein termed P-glycoprotein that is a non-specific, energy-dependent efflux pump. (See Roninson (ed)., 1991, Molecular and Cellular Biology of Multidrug Resistance in Tumor Cells, Plenum Press, N.Y., 1991; Gottesman et al., 1991, in Biochemical Bases for Multidrug Resistance in Cancer, Academic Press, N.Y., Chapter 11 for reviews). Examples of the latter type include topoisomerase II, the expression of which makes cells sensitive to the anticancer drug etoposide. Decreased expression of this enzyme makes neoplastic cells resistant to this drug. (See Gudkov et al., 1993, Proc. Natl. Acad. Sci. USA 90: 3231-3235). Although these are just single examples of the way that modulation of gene expression can influence chemotherapeutic drug sensitivity or resistance in neoplastic cells, these examples demonstrate the diagnostic and prognostic potential for identifying genes the expression of which (or the pattern of gene expression modulation thereof) are involved in mediating the clinical effectiveness of anticancer drug treatment.

Drug discovery programs have evolved to include rational therapeutic development strategies in addition to traditional empirical screening approaches. Rational therapy development focuses on the identification of specific pathways that are differentially activated in cancer cells compared to normal tissue (Bichsel et al., 2001, Cancer J. 7: 69-78; Winters, 2000, Curr. Opin. Mol. Ther. 2: 670-681). Such selective targeting can significantly reduce therapy-associated toxicity. Examples where this approach has led to the successful development of new anti-cancer agents include targeting HER2 with Herceptin (Bange et al., 2001, Nat. Med. 7: 548-552) in breast cancer and Gleevec (ST1571) inhibition of the BCR-abl kinase fusion protein in chronic myeloid leukemia (Brunstein & McGlave, 2001, Oncology (Huntingt) 15: 23-35).

Unfortunately, cancer specific pathways are not universal to the transformation process, which involves a variety of alterations in tumor suppressor genes, oncogenes, translocations, deletions and mutations. The genomic instability inherent to this pleiotropic background of metabolic alterations results in significant phenotypic heterogeneity within each tumor (Bertram, 2000, Mol. Aspects. Med. 21: 167-223; Yamasaki et al., 2000, Toxicol. Lett. 112-113: 251-256). Treatment targets are therefore unstable, leading to intrinsic and acquired resistance to rationally designed agents.

One class of genetic alterations that occur in neoplastic transformation is DNA amplification. Amplifications of genes or genetic loci are a common event in breast cancer that has useful clinical implications. For example, amplification of the HER2 gene on chromosome 17q11.2-12 is predictive of response to Herceptin therapy, and fluorescence in situ hybridization (FISH) detection kits are commercially available. Amplification correlates reasonably well with increased HER2 transcript levels. Gene amplification may be related to drug sensitivity or resistance to chemotherapeutic agents. Amplification of Epidermal Growth Factor receptor (EGFR) on chromosome 7p12 has been used to select patients who will benefit from Tarceva therapy. Amplification of Topoisomerase II-alpha (TOP2A) on chromosome 17q21-22 is also clinically relevant. TOP2A is a key enzyme in DNA replication, cell cycle progression and chromosome segregation, and is correlated with response to anthracyclines.

Several probes have recently been used to detect genetic changes that occur in cance; however, their biological significance has not been established. For example fluorescence in situ hybridization (FISH) probes have been developed to specific proteins such as c-myc and cyclin D (Heim, 1995, CANCER CYTOGENETICS, 2^{nd} Edition), In some cases, a FISH probe has been developed to detect a deletions For example, deletion of p53 and loss of heterozygosity has been detected in many tumor types. (Heim, 1995, Id.) Also, loss of p16, a putative tumor suppressor, can be detected by FISH (Balazs, 1997, Genes Chrom Cancer 19: 84-9; Okuda, 1995, Blood 85: 2321-30; Perry, 1997, J. Neuropath Exp Neuro 56: 999-1008; Sherr, 2000, Cancer Res 60: 3689-95) Lastly, loss of RB1, another tumor suppressor, can be detected by FISH (Heim, 1995, Id.). However, the use of this information to direct cancer care and/or therapy has not been established, and the manner in which these data affect clinical care and treatment is still in development.

In addition to these applications of FISH for diagnostic assays “targeted” to specific genes, it is known in the art to use FISH directed not to a specific gene but to a general region of a chromosome. Probes to general regions of interest have been produced but their use in cancer diagnosis and treatment has not been established. For example, chromosomal region 20q13 contains a putative oncogene, ZNF217, but the role of this protein in cancer progression or recurrence has not been established (Collins, 1998, Proc. Natl. Acad. Sci. USA 95: 8703-8, Tanner, 1994, Cancer Res 54: 4257-60) and thus the usefulness of using FISH to detect genetic changes in this region are unclear. Another example is a set of probes used to identify and diagnose oligodendrogliomas, consisting of probes to four chromosomal regions: 1p36, 1q25, 19p13, and 19q13. The clinical use of these probes is to first identify oligodendroglioma by the loss of both 1p36 and 19q13 and second, that glioblastoma tumors that have a deleted 1p36 may be more sensitive to certain types of chemotherapy. These four probes and their utility have been established in the clinic and are commercially available as a kit. These probes demonstrate the value of new DNA based diagnostics even if the exact gene(s) involved in drug response or tumor progression are not known.

An alternative methodology used to detect amplification of genetic loci in many types of cancer is comparative genomic hybridization (CGH), which has been used to determine the frequency of chromosomal amplifications. The relationship between such CGH data and cancer chemotherapy is in its infancy, however, and markers similar to HER2, EGFR, and TOP2A have not yet been identified for other important cancer treatment drugs such as Cisplatin and Carboplatin in ovarian cancer and non-small cell lung cancer, or Oxaliplatin in colorectal cancer. In addition, markers similar to the 1p/19q probe set have also not been developed for a wide range of tumor types and chemotherapy.

Thus, there is a need in this art for developing methods for identifying gene or genetic loci amplification that is predictive for clinical effectiveness of anticancer drug treatment therapies, in order to make more informed decisions for treating individual cancer patients with anticancer drugs having greatest likelihood of producing a positive outcome.

In contrast to the relative recent use of genetic approaches to identifying gene amplification and gene expression patterns that correlate with drug resistance or sensitivity, the art recognizes functional approaches to determining whether a specific cancer will respond to chemotherapeutic treatment. One example is the Extreme Drug Resistance (EDR®) assay. The EDR® Assay is an in vitro test that measures the ability of pharmaceutical agents and other chemotherapies to stop cancer cells from dividing and growing. This assay identifies patients that will not respond to a particular cancer therapeutic with >99% accuracy, and has been used in the art to exclude agents unlikely to be clinically effective from treatment of individual cancer patients, consequently sparing them the morbidity of ineffective chemotherapy. The EDR® Assay has also been used to select chemotherapeutic agents that have the greatest likelihood of being clinically effective, resulting in improved response rates and prolonged survival of cancer patients

Kern and Weisenthal (1990, J. Nat. Cancer Inst. 82: 582-588) showed that the EDR® Assay identified patients that would not respond to a cancer therapeutic. In this study, patients whose cancer cells demonstrated Extreme Drug Resistance to an anti-cancer drug had a greater than 99% failure rate when treated with that drug. In contrast, patients with Low Drug Resistance to an anti-cancer drug had a 52% clinical response rate when treated with that drug, which was significantly higher than the overall response rate to anti-cancer drugs of 29% (Id.).

In the practice of this assay, tumor cells are taken from a cancer biopsy and exposed to cancer chemotherapeutic agents in culture. During the culture period, radioactive thymidine is added, which is incorporated into the DNA of growing and dividing cancer cells (which are thus resistant to the cytostatic or cytotoxic effects of the cancer chemotherapeutic agent(s)). Tritiated thymidine is not incorporated into cells that are sensitive to the drug and reduce or suspend growth and division in response to the drug. Since cells affected by anticancer drugs do not divide, or divide more slowly, they therefore incorporate lesser amounts of the radioactive thymidine. By measuring the amount of radioactivity in a sample, the assay determines the relative resistance of an individual patient's cancer cells to a number of different chemotherapies.

The EDR® Assay is highly accurate at predicting clinically inactive drugs. Patients whose cancer cells have Extreme Drug Resistance to an anticancer agent have <1% response rate to that agent (Kern & Weisenthal, Id.). Clinical data suggest a therapeutic advantage in the activity of agents to which a tumor is highly active in vitro (Alberts et al., 1991, Anticancer Drugs 2:69-77), and treatment based on drugs that are active in vitro may improve response rates and survival (Kern & Weisenthal, Id.; Alberts et al., 1991, Anticancer Drugs 2:69-77; Kern, 1997, Cancer 79: 1447-50; Holloway et al., 2002, Gynecol Oncol 87: 8-16; Mehta et al., 2001, Breast Cancer Res. Treat 66: 225-37).

Thus, there is a need in this art to determine the relationship between functional drug resistance or sensitivity as evaluated, inter alia, by cell assays such as the EDR® Assay, and changes in gene expression or DNA copy number that correlate with the clinical resistance to a given therapeutic agent.

### SUMMARY OF THE INVENTION

This invention provides methods and reagents for identifying changes in DNA copy number comprising one or a plurality of genes amplified in tumor samples, most preferably human tumor samples, wherein the genes or genetic loci are differentially amplified in drug resistant versus drug sensitive tumor samples, particularly tumors resistant to platinum-based therapies and agents. The invention also provides methods for determining a prognosis for an individual having a tumor, particularly tumors resistant to platinum-based therapies and agents, wherein the prognosis is particularly directed towards determining the likelihood that a particular platinum-based treatment modality would be effective in treating the individual's cancer. The treatment modality is preferably a chemotherapeutic treatment, more preferably a platinum-based treatment, most preferably treatment with the anticancer drugs Cisplatin, Carboplatin, and Oxaliplatin. In certain embodiments the tumor is preferably a breast cancer tumor, colorectal cancer tumor, ovarian cancer tumor, or non-small cell lung cancer tumor. The invention also provides one or a plurality of said amplified of genetic loci that are altered for use in the prognostic methods of the invention.

Specific preferred embodiments of the present invention will become evident from the following more detailed description of certain preferred embodiments and the claims.

### BRIEF DESCRIPTION OF THE DRAWINGS

FIG. 1 represents a flowchart of an experimental procedure.

FIG. 2 is a graph demonstrating the similarity of the response of ovarian cancer tumors to both Cisplatin and Carboplatin. A total of over 30,000 ovarian tumors tested for both Cisplatin and Carboplatin resistance were compared (N=30,933). The Percent Inhibition (PCI, the amount the drug inhibits growth as compared to the control) for each drug was compared and correlated. The correlation was r=0.759. Thus, it can be expected that a probe that predicts resistance to either platinum based therapy can be used to predict both drug responses in ovarian cancer.

FIG. 3 is a graph demonstrating the similarity of the response of NSCLC to both Cisplatin and Carboplatin. A total of more than 1,600 lung tumors tested for both Cisplatin and Carboplatin resistance were compared (N=1,646). The Percent Inhibition (PCI, the amount the drug inhibits growth as compared to the control) for each drug was compared and correlated. The correlation was r=0.761. Thus, it can be expected that a probe that predicts resistance to either platinum based therapy can be used to predict both drug responses in lung cancer.

FIG. 4 is a graph demonstrating the similarity of the response of Colon cancer to both Cisplatin and Carboplatin. A total of about 500 colon tumors tested for both Cisplatin and Carboplatin resistance were compared (N=502). The Percent Inhibition (PCI, the amount the drug inhibits growth as compared to the control) for each drug was compared and correlated. The correlation was r=0.713. Thus, it can be expected that a probe that predicts resistance to either platinum based therapy can be used to predict both drug responses in lung cancer.

FIG. 5 is a graph demonstrating the response of colon cancer tumors to both Cisplatin and Oxaliplatin. A total of 170 colon tumors tested for both Cisplatin and Oxaliplatin resistance were compared (N=170). The Percent Inhibition (PCI, the amount the drug inhibits growth as compared to the control) for each drug was compared and correlated. The correlation was r=0.299. Thus, it can be expected that that different probes are required to predict resistance to Cisplatin and Oxaliplatin in colon cancer.

FIG. 6 is a graph demonstrating the difference in the adjusted DNA copy numbers (tumor DNA copy numbers divided by NHDF DNA copy number).

### DETAILED DESCRIPTION OF THE PREFERRED EMBODIMENTS

This invention provides methods for making a prognosis about disease course in a human cancer patient. For the purposes of this invention, the term “prognosis” is intended to encompass predictions and likelihood analysis of disease progression, particularly tumor recurrence, metastatic spread and disease relapse and most particularly resistance to chemotherapeutic agents, particularly platinum-based agents, commonly used in treating cancer. The prognostic methods of the invention are intended to be used clinically in making decisions concerning treatment modalities, specifically treatment modalities encompassing choices relating to chemotherapeutic treatment, particularly platinum-based therapies and choosing chemotherapeutic agents, particularly platinum-based agents, for which a particular tumor in a patient is not inherently resistant or likely to become resistant, as well as including therapeutic intervention, diagnostic criteria such as disease staging, and disease monitoring and surveillance for metastasis or recurrence of neoplastic disease.

As provided herein, the methods of this invention are directed towards determining resistance of a tumor sample, most preferably a human tumor sample, to chemotherapeutic drugs. In certain embodiments, these drugs are platinum based and DNA damaging agents, including but not limited to Cisplatin, Carboplatin, Oxaliplatin, Araplatin, and Satraplatin, but most preferably Cisplatin, Carboplatin, and Oxaliplatin. However, the disclosure set forth herein is intended to encompass any chemotherapeutic drug that induces amplification of genetic loci, and any tumor sample, most preferably any human tumor sample, comprising cells whose chromosomal DNA comprises amplified genetic loci.

The invention also provides methods for identifying genetic loci useful for making a cancer prognosis, by virtue of said genetic loci being differentially amplified in tumors, particularly tumors resistant to platinum-based therapies. The invention also provides a plurality of said genetic loci that can be employed in the prognostic methods of the invention individually or in combination to develop a prognosis, more particularly a therapeutic prognosis and most particularly a clinical decision regarding chemotherapy and chemotherapeutic drug choices for an individual patient, particularly relating to treatment using platinum-based therapies and agents.

The invention therefore provides methods for individualized, genetic-based medicine by informing a caregiver of the likelihood of successful treatment of an individual patient with a treatment modality, particularly platinum-based treatment modalities including but not limited to Cisplatin, Carboplatin, and Oxaliplatin.

The methods of the invention are preferably performed using human cancer patient tumor samples, most preferably samples are preserved, for example in paraffin, and prepared for histological and immunohistochemical analysis.

For the purposes of this invention, the term “tumor sample” is intended to include resected solid tumors, biopsy material, pathological specimens, bone marrow aspirates, and blood samples comprising neoplastic cells of hematopoietic origin, as well as benign tumors, particularly tumors of certain tissues such as brain and the central nervous system. Most particularly, the tumor samples of this invention are breast cancer tumor samples, colon cancer tumor samples, ovarian tumor samples, or non-small cell lung (NSCLC) tumor samples.

Also included in the tumor samples of the invention are samples that have been treated or manipulated after resection to increase the proportion of tumor cells in the sample. Examples of such treatments include physical and/or enzymatic dissociation of the tumor sample and differential recovery or separation of the tumor cells from non-tumor cells (such as stromal cells, hematopoietic cells, or non-tumor tissue cells resulting, inter alia, from resection at the margins of the tumor). Tumor cell separation can be achieved using differential growth methods (in culture or in semisolid medium, for example) or by specifically or differentially labeling tumor cells and separating them thereby. For example, detectably-labeled immunological reagents (including antibodies, particularly monoclonal antibodies, or immunospecific fragments thereof) are used to specifically or differentially label tumor cells, which are then separated from non-tumor cells on the basis of their specific or differential labeling. Detectable labels include, for example, fluorescent, antigenic, radioisotopic or biotin labels, among others. Alternatively, labeled secondary or tertiary immunological detection reagents can be used to detect binding of the neoplastic immunological reagents (i.e., in secondary antibody (sandwich) assays). Separation methods include, for example, immunoaffinity columns, immunomagnetic beads, fluorescence activated cell sorting (FACS), most preferably FACS.

Examples of immunological reagents useful in the practice of particular aspects of this invention include antibodies, most preferably monoclonal antibodies, that recognize tumor antigens, including but not limited to CA15-3 (breast cancer), CA19-9 (gastrointestinal cancer), CA125 (ovarian cancer), CA242 (gastrointestinal cancer), p53 (colorectal cancer), prostate-specific acid phosphatase (prostate cancer), Rb (retinoblastoma), CD56 (small cell lung cancer), prostate-specific antigen (PSA, prostate cancer), carcinoembryonic antigen (CEA), melanoma antigen and melanoma-associated antigens (melanoma), mucin-1 (carcinoma), HER2 (breast cancer), and EGFR (breast and ovarian cancer). Preferred immunological reagents recognize breast cancer, including but not limited to CA15-3, HER2 and EGFR.

The immunological reagents of the invention are preferably detectably-labeled, most preferably using fluorescent labels that have excitation and emission wavelengths adapted for detection using commercially-available instruments such as and most preferably fluorescence activated cell sorters (FACS). Examples of fluorescent labels useful in the practice of the invention include phycoerythrin (PE), fluorescein isothiocyanate (FITC), rhodamine (RH), Texas Red (TX), Cy3, Hoechst 33258, and 4′,6-diamidino-2-phenylindole (DAPI). Such labels can be conjugated to immunological reagents, such as antibodies and most preferably monoclonal antibodies using standard techniques (Maino et al., 1995, Cytometry 20: 127-133).

As used herein, the terms “microarray,” “bioarray,” “biochip” and “biochip array” refer to an ordered spatial arrangement of immobilized biomolecular probes arrayed on a solid supporting substrate. Biochips, as used in the art, encompass substrates containing arrays or microarrays, preferably ordered arrays and most preferably ordered, addressable arrays, of biological molecules that comprise one member of a biological binding pair. Typically, such arrays are oligonucleotide arrays comprising a nucleotide sequence that is complementary to at least one sequence that may be or is expected to be present in a biological sample, either RNA or DNA. Alternatively, and preferably, proteins, peptides or other small molecules can be arrayed in such biochips for performing, inter alia, immunological analyses (wherein the arrayed molecules are antigens) or assaying biological receptors (wherein the arrayed molecules are ligands, agonists or antagonists of said receptors). Useful microarrays for detecting differential gene expression between chemotherapeutic drug sensitive and resistant neoplastic cells are described, inter alia, in U.S. Pat. No. 6,040,138 to Lockhart et al. (commercially-available from Affymetrix, Inc., Santa Clara, Calif.) and U.S. Pat. No. 6,004,755 to Wang (commercially-available from Incyte Inc., Palo Alto, Calif.) and are also commercially available, inter alia, from Research Genetics (Huntsville, Ala.). Non-limiting examples of commercially available biochips useful in the practice of the invention are the Affymetrix GeneChip® Human Genome U133 Set (which includes both HG-U133A and HG-U133B), and the Affymetrix 50KXbaI microarray.

As used in certain aspects of the methods of the invention, gene arrays or microarrays comprise a solid substrate, preferably within a square of less than about 10 microns by 10 microns on which a plurality of positionally-distinguishable polynucleotides are attached. These probe sets can be arrayed onto areas of up to 1 to 2 cm², providing for a potential probe count of >30,000 per chip. The solid substrate of the gene arrays can be made out of silicon, glass, plastic or any suitable material. The form of the solid substrate may also vary and may be in the form of beads, fibers or planar surfaces. The sequences of these polynucleotides are determined from tumor-specific gene sets identified by analysis of gene expression profiles from a plurality of tumors as described above. The polynucleotides are attached to the solid substrate using methods known in the art (see, for example, DNA MICROARRAYS: A PRACTICAL APPROACH, Schena, ed., Oxford University Press: Oxford, UK, 1999) at a density at which hybridization of particular polynucleotides in the array can be positionally distinguished. Preferably, the density of polynucleotides on the substrate is at least 100 different polynucleotides per cm², more preferably at least 300 polynucleotides per cm². In addition, each of the attached polynucleotides comprises at least about 25 to about 50 nucleotides and has a predetermined nucleotide sequence. Larger nucleotides generated from BACs can be also be used and each of these has a predetermined sequence that is complementary to human genomic DNA. Target RNA, cDNA, or DNA preparations are used from tumor samples that are complementary to at least one of the polynucleotide sequences on the array and specifically bind to at least one known position on the solid substrate.

Gene expression analysis is performed to detect differences in gene expression between neoplastic cells that are sensitive to a cytotoxic, chemotherapeutic drug, particularly a platinum-based drug including but not limited to Cisplatin, Carboplatin, and Oxaliplatin, and drug resistant neoplastic cells. RNA from the drug resistant neoplastic cells and drug sensitive neoplastic cells is individually isolated and cDNA prepared therefrom. In preferred embodiments, the cDNA is detectably labeled, for example using radioactively-labeled or fluorescently-labeled nucleotide triphosphates. Hybridization of gene expression microarrays produces patterns of gene expression specific for cytotoxic, chemotherapeutic drug resistant neoplastic cells and neoplastic cells sensitive to the same drug, particularly a platinum-based drug including but not limited to Cisplatin, Carboplatin, and Oxaliplatin. Identification of genes and patterns of genes differentially expressed in these cells is established by comparison of the gene expression pattern obtained by performing the microarray hybridization analysis on cDNA from neoplastic cells that are resistant to and sensitive to the cytotoxic, chemotherapeutic drug, particularly a platinum-based drug including but not limited to Cisplatin, Carboplatin, and Oxaliplatin. Advantageously, tumor samples from human patients and breast cancer cell lines sensitive or resistant to platinum-based drugs including but not limited to Cisplatin, Carboplatin, and Oxaliplatin, are compared using bioinformatics analysis to identify genes statistically correlated with drug resistance or sensitivity.

Once identified, differentially amplified and/or overexpressed genes can be used alone or in combination to assay individual tumor samples and determine a prognosis, particularly a prognosis regarding treatment decisions, most particularly regarding decisions relating to treatment modalities such as chemotherapeutic treatment, particularly a platinum-based treatment modality including but not limited to Cisplatin, Carboplatin, and Oxaliplatin.

Comparative Genomic Hybridization (CGH) is a technique for detecting mutations at the chromosomal level. Genomic DNA is purified from cells and labeled with fluorescent dyes. The labeled DNA is then hybridized to immobilized bacterial artificial chromosomes (BACs) or specific probes to regions of DNA. BACs are artificially constructed chromosomes made from bacterial DNA and include inserted segments of 100,000-300,000 base pairs from human DNA. The probes used in these experiments consisted of oligonucleotides comprising twenty nucleotides whose sequence was matched to human DNA. Unlike the gene expression arrays, the sequences used for DNA probes are made to genomic DNA and represent a valuable tool for the analyses of DNA copy number in human tumors. The resulting signals from the hybridization are analyzed for alterations in DNA gains and losses as compared to a standard human genome.

Once identified, differentially amplified or deleted genes or genetic loci can be used alone or in combination to assay individual tumor samples and determine a prognosis, particularly a prognosis regarding treatment decisions, most particularly regarding decisions relating to treatment modalities such as chemotherapeutic treatment, particularly a platinum-based treatment including but not limited to Cisplatin, Carboplatin, and Oxaliplatin. These changes can be detected with such procedures as Fluorescence In Situ Hybridization (FISH), Chromogenic In Situ Hybridization (CISH), DNA microarrays, or CGH arrays.

The methods of the invention for identifying genetic loci amplified in drug resistant tumor cells, particularly tumors resistant to a platinum-based drug including but not limited to Cisplatin, Carboplatin, and Oxaliplatin, comprise comparing the levels of drug resistance, particularly resistance to platinum-based drugs including but not limited to Cisplatin, Carboplatin, and Oxaliplatin, as determined by EDR® assay with differential gene amplification. Differential gene amplification was first compared to the average levels of DNA found to be four normal diploid fibroblasts (i.e., non-cancerous cells with a cytogenetically-normal diploid genotype. If the results from the DNA Mapping arrays indicated that there are higher levels of DNA copy number in the normal fibroblasts than two, then that number was used to compare with the tumors. The reason for using the higher number to compare with tumor DNA copy number was because variations in copy number may have represented artifacts or other experimental issues with the probes on the array and not be truly representative of the actual DNA copy number. Once regions of DNA that appeared to be amplified were identified in the tumors, the results for these regions were compared between platinum-based drug resistant and platinum-based drug responsive tumors. If the amplicons were present in only drug resistant tumors, the frequency of this event was analyzed. If the increase in DNA copy number was greater then five and present in at least one quarter of tumors analyzed, it was considered a potential drug resistance marker and further evaluated using other technologies.

The most direct method to discover genetic loci that vary between platinum-based drug resistant and drug sensitive tumors is through CGH screening. Purification of DNA is done using any of several commercially available kits (such as those produced by Qiagen, Chatsworth, Calif. and Promega, Madison, Wis.).

In these experiments, DNA from ovarian tumors was purified as follows. A commercially-available DNA Extraction kit (Gentra, Minneapolis, Minn.) was utilized to isolate intact genomic DNA from tumor explants and primary cell lines, as per manufacture's recommendation. Briefly, approximately one million cells were pelleted by centrifugation at 13,000×g for 1 minute and the media decanted. The cell pellet was vortexed and resuspended in the residual media (ca. 20 microlitre, μL). 250 microlitre of lysis buffer (containing RNaseA and Proteinase K) was added and rapidly mixed to achieve confluent lysis and then incubates at 37° C. for 30 minutes. A protein precipitation solution provided by the kit manufacturer (100 μL) was added and mixed by vortexing vigorously. Cellular debris was pelleted by centrifugation at 13,000×g for 5 minutes. The supernatant was removed to a fresh tube containing 800 μL ice cold absolute ethanol and DNA was precipitated by rocking the tube back and forth until the precipitated DNA was visualized. The precipitated DNA was removed to a fresh tube and air dried for 10 minutes, and then resuspended in an adequate volume of DNA Hydration Solution to generate 500 to 1000 μg/mL DNA (final concentration). DNA rehydration was facilitated by incubation at room temperature for an additional 60 to 120 minutes.

The protocols provided by Affymetrix were followed as detailed in the Assay Manual with one major exception. In our experiments, we did not perform the PCR steps to generate DNA for labeling and testing. Instead, we purified DNA as detailed above and used 100 μg of DNA beginning with Step 7: Fragmentation (as indicated in the Assay Manual, pg 45). The DNA was added to Fragmentation Reagent and Fragmentation Buffer on ice, vortexed, and run in a thermocycler using the following program: 37° C. for 35 minutes, 95° C. for 15 minutes, and held at 4° C. until removed. A sample was run on an agar gel to confirm that fragmentation had occurred. The protocol was then followed as detailed in the Assay Manual for Labeling, Hybridization, and Washing.

Amplification of DNA from a chromosomal regions identified as described herein may be used to predict resistance to platinum-based therapies, including but not limited to Cisplatin, Carboplatin, and Oxaliplatin, and consequent poor prognosis in multiple tumor types, including breast cancer, colon cancer, ovarian cancer, and non-small cell lung cancer (NSCLC). In preferred embodiments, the chromosomal regions amplified in Cisplatin, Carboplatin, and Oxaliplatin resistant tumors include human chromosomes 1q41, 3q13, 3q26, 8p23, 8q13, 13q33, and 16q23. These changes may be used individually or in combination to predict drug response. These changes in DNA can be used to determine resistance to platinum-based agents or therapies in breast cancer, colon cancer, ovarian cancer, or non-small cell lung cancer.

In addition, amplifications at the following genetic locations are selective and predictive for Cisplatin and Carboplatin resistance. These changes include 1q41, 2p12, 3p24, 3q13, 3q25, 3q26, 4q21, 4q32, 5q33, 7q32, 8p23, 8q13, 8q24, 9q31, 10p14, 11p13, 11p15, 12q21, 12q22, 12q23, 13q13, 13q33, 15q25, 16p13, 16q23, 17p13, and 19p12. These changes may be used individually or in any combination to predict drug response to platinum based therapy in ovarian and NSCLC.

In addition, amplifications at the following genetic locations are selective and predictive for Oxaliplatin resistance in colon cancer. These changes include 1p31, 1q24, 1q41, 2q22, 3p12, 3p26, 3q13, 3q22, 3q26, 4p15, 4q13, 5p13, 5q15, 5q23, 6p12, 6q16, 6q21, 6q24, 7p21, 7q35, 8p22, 8p23, 8q13, 8q23, 8q24, 9p23, 9q33, 11p14, 11q14, 12q21, 13q31, 13q33, 14q22, 14q31, 16q23, 18q12, 18q22, 20p12, and 21q21. These changes may be used individually or in any combination to predict drug response to Oxaliplatin in Colon cancer.

The description set forth above and the Examples set forth below recite exemplary embodiments of the invention. The following Examples are intended to further illustrate certain preferred embodiments of the invention and are not limiting in nature.

### EXAMPLES

### Example 1

### The EDR Assay

### Tumor Specimen Handling

Viable tumors samples were obtained from patients with malignant disease and placed into Oncotech transport media (complete medium, RPMI supplemented with 3% Fetal Calf Serum and antibiotics, as described below in the section Tissue Culture and Expansion). Sample collection and preparation were performed by personnel at the referring institution immediately after collection and shipped by overnight courier where the tumor's in vitro drug response profile was determined. Upon receipt, the tumor was processed as follows: three areas of the tumor were removed from the sample, fixed in Formalin, paraffin embedded, sectioned and hematoxylin and eosin stained for pathologists' review to ensure agreement with the referring institution histological diagnosis. After in vitro drug response of the tumor specimens were determined by the laboratory, this information was sent back to the treating physician to aid in patient treatment selection.

The remainder of the sample is disaggregated mechanically and processed into a cell suspension for the Extreme Drug Resistance (EDR) assay. A cytospin preparation from a single cell suspension of the tumor was examined by a technologist to determine the presence and viability of malignant cells in the specimen.

### EDR Assay

The EDR assay is an agarose-based culture system, using tritiated thymidine incorporation to define in vitro drug response. This assay is predictive of clinical response (Kern et al., 1990, “Highly specific prediction of antineoplastic resistance with an in vitro assay using suprapharmacologic drug exposures,” J. Nat. Cancer Inst. 82: 582-588). Tumors were cut with scissors into pieces of 2 mm or smaller in a Petri dish containing 5 mL of complete medium. The resultant slurries were mixed with complete media containing 0.03% DNAase (2650 Kunitz units/mL) and 0.14% collagenase I (both enzymes obtained from Sigma Chemical Co., St. Louis, Mo.), placed into 50 mL Erlenmeyer flasks with stirring, and incubated for 90 min at 37° C. under a humidified 5% CO₂ atmosphere. After enzymatic dispersion into a near single cell suspension, tumor cells were filtered through nylon mesh, and washed in complete media. A portion of the cell suspension was used for cytospin slide preparation and stained with Wright-Giemsa for examination by a medical pathologist in parallel with Hematoxylin-Eosin stained tissue sections, to confirm the diagnosis and to determine the tumor cell count and viability. Tumor cells were then suspended in soft agarose (0.13%) and plated at 20,000-50,000 cells per well in 24-well plates wherein each well contained an agarose underlayer (0.4%). Tumor cells were incubated under conventional cell culture conditions for 5 days in the presence or absence of 1.67 μM Cisplatin, 10.26 μM Carboplatin, or 2.52 μM Oxlaplatin. Cells were pulsed with tritiated thymidine (New Life Science Products, Boston, Mass.) at 5 μCi per well for the last 48 hours of the culture period. After labeling, cell culture plates were heated to 96° C. to liquify the agarose, and the cells are harvested with a micro-harvester (Brandel, Gaithersburg, Md.) onto glass fiber filters. The radioactivity trapped on the filters was counted with an LS-6500 scintillation Counter (Beckman, Fullerton, Calif.). Untreated cells served as a negative control. In the positive (background) control group, cells were treated with a supratoxic dose of Cisplatin (33 μM), sufficient to cause death of all the cells. Detectable radioactivity for this group was considered non-specific background related to debris trapping of tritiated thymidine on the filter. After subtracting background control values, percent control inhibition (PCI) of proliferation was determined by comparing thymidine incorporation by the treatment group with incorporation by the negative control group, using the formula:

PCI=100%×{1−(CPM treatment group/CPM control group)}.

The determination of drug effects on tumor proliferation is performed in duplicate.

Specimens were classified as EDR (extreme drug resistant), to Cisplatin if the PCI was less than 55%. Specimens were classified as LDR (low drug resistance) to Cisplatin if the PCI was greater than 76%. Specimens were classified as EDR to Carboplatin if the PCI was less than 48%. Specimens were classified as LDR to Carboplatin if the PCI was greater than 67% Specimens were classified as EDR to Oxaliplatin if the PCI was less than 51%. Specimens were classified as LDR to Oxaliplatin if the PCI was greater than 90%.

### Example 2

### Gene Array Analysis

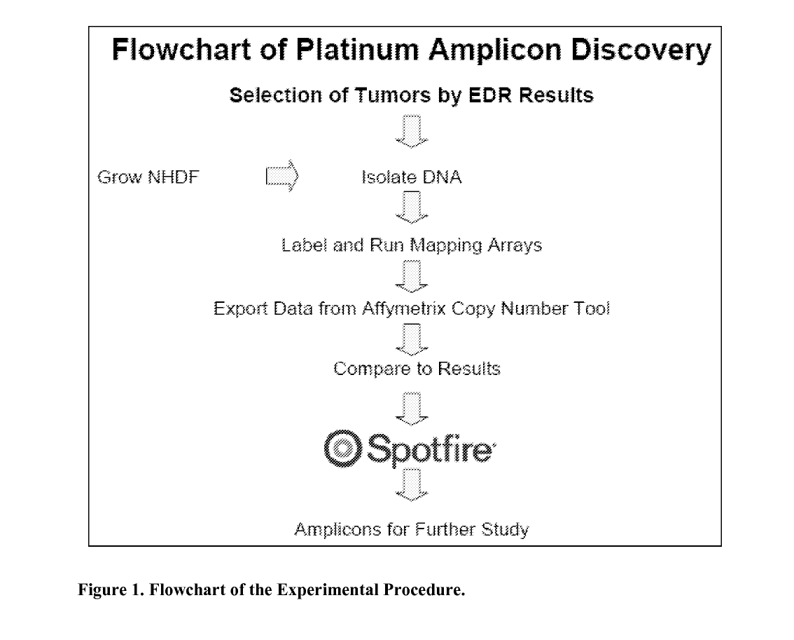

Ovarian tumors for use in the gene array were selected based on the results for platinum drugs in the EDR assay. Tumors that were either EDR to Cisplatin or LDR to Cisplatin were chosen for comparison. DNA was extracted and purified from these tumors as described below (FIG. 1). A commercially-available DNA Extraction kit (Gentra, Minneapolis, Minn.) was utilized to isolate intact genomic DNA, as per the manufacture's recommendation. Normal human diploid fibroblasts (NHDFs) were used as the control and DNA from the samples were purified the same way as done with the tumors. Briefly, approximately one million cells were pelleted by centrifugation at 13,000×g for 1 minute and the media was decanted. The cell pellet was vortexed and resuspended in the residual media (ca. 20 μL). 250 μL of lysis buffer (containing RNaseA and Proteinase K) was added and rapidly mixed to achieve confluent lysis and then incubated at 37° C. for 30 minutes. A protein precipitation solution provided by the kit manufacturer (100 μL) was added and mixed by vortexing vigorously. Cellular debris was pelleted by centrifugation at 13,000×g for 5 minutes. The supernatant was removed to a fresh tube containing 800 μL ice cold absolute ethanol, and the DNA was precipitated by rocking the tube back and forth until the precipitated DNA was visualized. The precipitated DNA was removed to a fresh tube and air dried for 10 minutes, and then resuspended in an adequate volume of DNA Hydration Solution obtained from the manufacturer to generate 500 to 1000 μg/mL DNA (final concentration). DNA rehydration was facilitated by incubation at room temperature for an additional 60 to 120 minutes.

Purified DNA was subsequently amplified and labeled following the manufacture's Assay Manual instructions with slight modification. DNA for labeling and testing was not generated by PCR, but rather the DNA was purified DNA as detailed above, and 100 μg of DNA was used with the Fragmentation step (step 7 in the Assay Manual (pg 45)). The DNA was added to Manufacturer's Fragmentation Reagent and Fragmentation Buffer on ice, vortexed, and amplified in a thermocycler using the following program: 37° C. for 35 minutes, 95° C. for 15 minutes, and held at 4° C. until removed. Fragmentation was confirmed by running a sample on an agar gel.

Genomic DNA purified and fragmented as described above was then hybridized to an Affymetrix 50K XbaI mapping array according to Manufacturer's instructions and hybridization results obtained and analyzed as described below.

### Example 3

### Data Analyses

The raw Mapping Array data obtained from the experiments described above was analyzed in the Affymetrix Chromosome Copy Number Analysis Tool Version 2.1.0.1. The results generated in the Affymetrix Chromosome Copy Number Analysis Tool from arrays of NHDFs were used as controls to standardize the results obtained from tumor sample DNA. Since NHDFs are “normal” they were expected to contain no more than two copies of each chromosome or chromosome locus. Any deviation from a copy number of two from NHDF samples was attributed to probe variation and used to standardize results from tumor samples, including samples found by EDR assay to be either LDR or EDR to platinum based drugs.

An example of the data from the Affymetrix Mapping Array is shown below:

| | Probe | | Physical | 719 | 719 | 719 | 719 | 719 | 719 |
|---|---|---|---|---|---|---|---|---|---|
| | Set | Chromosome | Position | S_Call | S_SPA_CN | S_SPA_pVal | S_GSA_CN | S_GSA_PVAL | S_LCN |
| 1 | SNP_A- | 1 | 2266413 | NoCall | 13.2 | 20 | 13.2 | 20 | 0 |
| | 1718890 | | | | | | | | |
| 2 | SNP_A- | 1 | 3118283 | NoCall | 3.75 | 2.43 | 2.11 | 0.8 | 0 |
| | 1668776 | | | | | | | | |
| 3 | SNP_A- | 1 | 3188424 | NoCall | 1.59 | −0.87 | 2.11 | 0.8 | 0 |
| | 1723597 | | | | | | | | |
| 4 | SNP_A- | 1 | 3220904 | NoCall | 0.67 | −6.06 | 2.11 | 0.8 | 0 |
| | 1728870 | | | | | | | | |
| 5 | SNP_A- | 1 | 3326028 | NoCall | 1.15 | −2.8 | 2.01 | 0.91 | 0 |
| | 1669946 | | | | | | | | |
| 6 | SNP_A- | 1 | 3327493 | NoCall | 2.25 | 0.43 | 2.01 | 0.91 | 0 |
| | 1708099 | | | | | | | | |
| 7 | SNP_A- | 1 | 3362496 | NoCall | 3.29 | 2.22 | 2 | 0.91 | 0 |
| | 1716798 | | | | | | | | |
| 8 | SNP_A- | 1 | 3568769 | AA | 1.4 | −1.6 | 1.84 | −0.57 | 3.5 |
| | 1701872 | | | | | | | | |
| 9 | SNP_A- | 1 | 3744122 | NoCall | 1.41 | −0.8 | 1.56 | −0.93 | 3.5 |
| | 1697748 | | | | | | | | |
| 10 | SNP_A- | 1 | 3744475 | BB | 1.18 | −1.84 | 1.56 | −0.93 | 3.5 |
| | 1699138 | | | | | | | | |
| 11 | SNP_A- | 1 | 3788581 | AA | 2.23 | 0.48 | 1.56 | −0.92 | 3.5 |
| | 1750020 | | | | | | | | |
| 12 | SNP_A- | 1 | 4082159 | NoCall | 1.01 | −4.37 | 1.01 | −4.37 | 3.5 |
| | 1646481 | | | | | | | | |
| 13 | SNP_A- | 1 | 4388284 | NoCall | 0.9 | −8.69 | 1.48 | −3.48 | 3.5 |
| | 1701882 | | | | | | | | |
| 14 | SNP_A- | 1 | 4388412 | NoCall | 1.55 | −1.92 | 1.48 | −3.48 | 3.5 |
| | 1701982 | | | | | | | | |
| 15 | SNP_A- | 1 | 4496979 | NoCall | 0.86 | −3.9 | 1.49 | −3.46 | 3.5 |
| | 1749896 | | | | | | | | |
| 16 | SNP_A- | 1 | 4497550 | BB | 3.19 | 2.79 | 1.49 | −3.46 | 3.5 |
| | 1750090 | | | | | | | | |
| 17 | SNP_A- | 1 | 4562378 | NoCall | 0.91 | −5.66 | 1.44 | −3.46 | 3.5 |
| | 1714480 | | | | | | | | |
| 18 | SNP_A- | 1 | 4789244 | NoCall | 1.16 | −3.56 | 3.86 | 3.21 | 3.5 |
| | 1649717 | | | | | | | | |
| 19 | SNP_A- | 1 | 4874378 | NoCall | 7.41 | 20 | 3.86 | 2.7 | 3.5 |
| | 1703834 | | | | | | | | |
| 20 | SNP_A- | 1 | 5001088 | NoCall | 7.7 | 4.7 | 3.01 | 0.75 | 3.5 |
| | 1695724 | | | | | | | | |
| 21 | SNP_A- | 1 | 5035232 | NoCall | 2 | −0.32 | 2.89 | 0.8 | 3.5 |
| | 1693756 | | | | | | | | |
| 22 | SNP_A- | 1 | 5065682 | NoCall | 0.8 | −8.27 | 3.02 | 0.63 | 3.5 |
| | 1690704 | | | | | | | | |
| 23 | SNP_A- | 1 | 5147985 | NoCall | 0.36 | −12.42 | 2.58 | 2.6 | 3.5 |
| | 1755813 | | | | | | | | |
| 24 | SNP_A- | 1 | 5163918 | NoCall | 3.26 | 1.68 | 2.58 | 2.61 | 3.5 |
| | 1747364 | | | | | | | | |
| 25 | SNP_A- | 1 | 5164037 | BB | 2.42 | 0.66 | 2.58 | 2.61 | 3.5 |
| | 1747266 | | | | | | | | |
| 26 | SNP_A- | 1 | 5164291 | NoCall | 3.21 | 1.07 | 2.58 | 2.61 | 3.5 |
| | 1747118 | | | | | | | | |
| 27 | SNP_A- | 1 | 5168527 | BB | 0.64 | −16.26 | 2.57 | 2.61 | 3.5 |
| | 1745353 | | | | | | | | |
| 28 | SNP_A- | 1 | 5168929 | NoCall | 3.9 | 4 | 2.57 | 2.61 | 3.5 |
| | 1745179 | | | | | | | | |
| 29 | SNP_A- | 1 | 5251605 | NoCall | 1.66 | −0.55 | 2.04 | 3.37 | 3.5 |
| | 1755067 | | | | | | | | |
| 30 | SNP_A- | 1 | 5562178 | NoCall | 4.52 | 4.32 | 2.17 | 0.93 | 3.5 |
| | 1703272 | | | | | | | | |
| 31 | SNP_A- | 1 | 5633741 | NoCall | 2.61 | 0.95 | 2.15 | 0.97 | 3.5 |
| | 1652197 | | | | | | | | |
| 32 | SNP_A- | 1 | 5672766 | NoCall | 1.43 | −1.75 | 2.15 | 0.98 | 3.5 |
| | 1705830 | | | | | | | | |
| 33 | SNP_A- | 1 | 5678003 | NoCall | 2.19 | 0.57 | 2.14 | 0.99 | 3.5 |
| | 1743804 | | | | | | | | |
| 34 | SNP_A- | 1 | 5679187 | NoCall | 0.66 | −8.78 | 2.14 | 0.99 | 3.5 |
| | 1744186 | | | | | | | | |
| 35 | SNP_A- | 1 | 5679229 | NoCall | 1.53 | −1.07 | 2.14 | 0.99 | 3.5 |
| | 1744304 | | | | | | | | |
| 36 | SNP_A- | 1 | 5940838 | NoCall | 0.93 | −2.95 | 2.74 | 1.14 | 3.5 |
| | 1698066 | | | | | | | | |
| 37 | SNP_A- | 1 | 5941712 | NoCall | 1.45 | −0.84 | 2.74 | 1.14 | 3.5 |
| | 1698244 | | | | | | | | |
| 38 | SNP_A- | 1 | 5977200 | NoCall | 4.46 | 2.92 | 2.74 | 1.15 | 3.5 |
| | 1743594 | | | | | | | | |
| 39 | SNP_A- | 1 | 6037313 | NoCall | 4.14 | 5.5 | 2.76 | 1.18 | 3.5 |
| | 1676349 | | | | | | | | |
| 40 | SNP_A- | 1 | 6310488 | NoCall | 4.98 | 1.72 | 4.98 | 1.72 | 3.5 |
| | 1673751 | | | | | | | | |
| 41 | SNP_A- | 1 | 6666834 | NoCall | 1.34 | −2.26 | 1.85 | −0.77 | 3.5 |
| | 1691957 | | | | | | | | |
| 42 | SNP_A- | 1 | 6698909 | NoCall | 3.42 | 3.31 | 1.85 | −0.78 | 3.5 |
| | 1699284 | | | | | | | | |
| 43 | SNP_A- | 1 | 6698929 | NoCall | 1.24 | −1.45 | 1.85 | −0.78 | 3.5 |
| | 1699392 | | | | | | | | |
| 44 | SNP_A- | 1 | 6872037 | AA | 1.37 | −2.85 | 1.84 | −0.83 | 3.5 |
| | 1684395 | | | | | | | | |
| 45 | SNP_A- | 1 | 7186927 | NoCall | 5.4 | 3.9 | 3.34 | 1.93 | 3.5 |
| | 1683539 | | | | | | | | |
| 46 | SNP_A- | 1 | 7187335 | NoCall | 1.62 | −0.88 | 3.34 | 1.93 | 3.5 |
| | 1683663 | | | | | | | | |
| 47 | SNP_A- | 1 | 7297576 | NoCall | 2.99 | 2.78 | 3.33 | 1.94 | 3.5 |
| | 1739552 | | | | | | | | |
| 48 | SNP_A- | 1 | 7695267 | NoCall | 1.94 | −0.35 | 4.05 | 4 | 3.5 |
| | 1682335 | | | | | | | | |
| 49 | SNP_A- | 1 | 7703744 | NoCall | 4.95 | 20 | 4.05 | 3.99 | 3.5 |
| | 1684343 | | | | | | | | |

Column, “719_S” refers to a unique identifier for the tumor used in the experiment. The data columns are: “Probe Set,” which refers to the Affymetrix identifier for that probe, “Chromosome,” which is the chromosome on which the probe is located, “Physical Position,” which refers to the mapping location within the human genome, “Call,” which refers to the decision made by the Affymetrix if a SNP is present in this probe (this column is not used in these analyses), “SPA_CN,” which refers to the Probe Specific Average which is a technique the Affymetrix software uses to examine the probe and its immediate surrounding probes to determine if there is an increase in signal (i.e. DNA copy number), “SPA_pVAL,” which is a measure of how strong the DNA copy change is and in what direction the change occurs (a positive value is increase and a negative value is a loss), “GSA_CN,” which refers to a copy number (i.e., DNA content at that location) as a Genome Smoothed Average (a method of data analysis whereby the signal from the probe is compared to the entire genome to generate a copy number), and the “GSA pVAL,” which is a measure of how strong the DNA copy change is and in what direction the change occurs (a positive value is increase and a negative value is a loss). These data were created by the Affymetrix Chromosome Copy Number Analysis Tool Version 2.1.0.1. Whereas the use of GSA analyses is not meant to be limiting, the genome-wide average is advantageous in that it is representative of the DNA content in the tumor, especially because many tumors can become aneuploid as they progress.

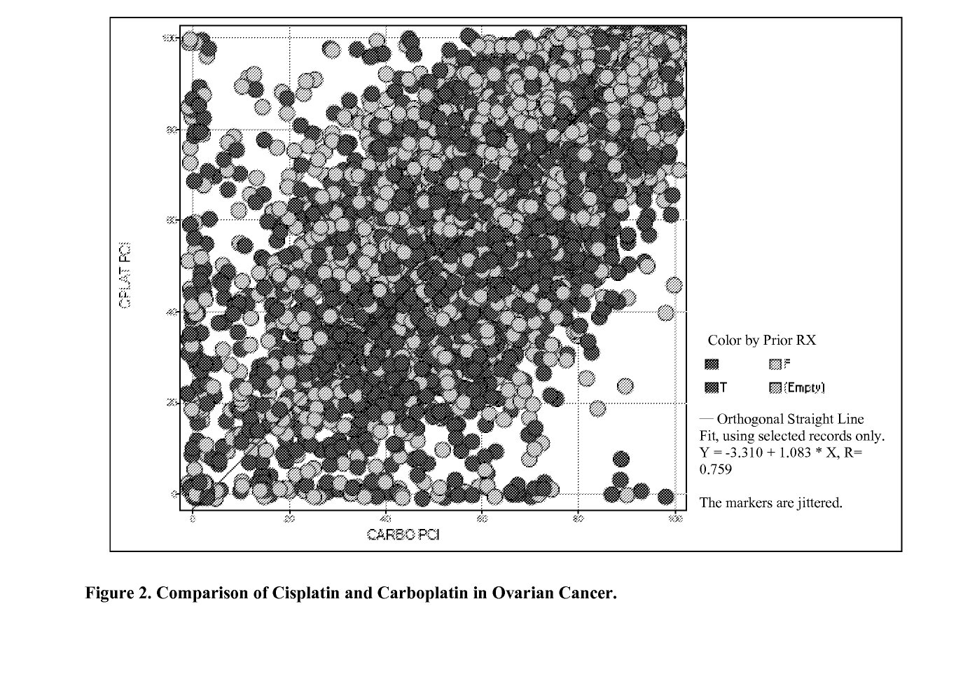

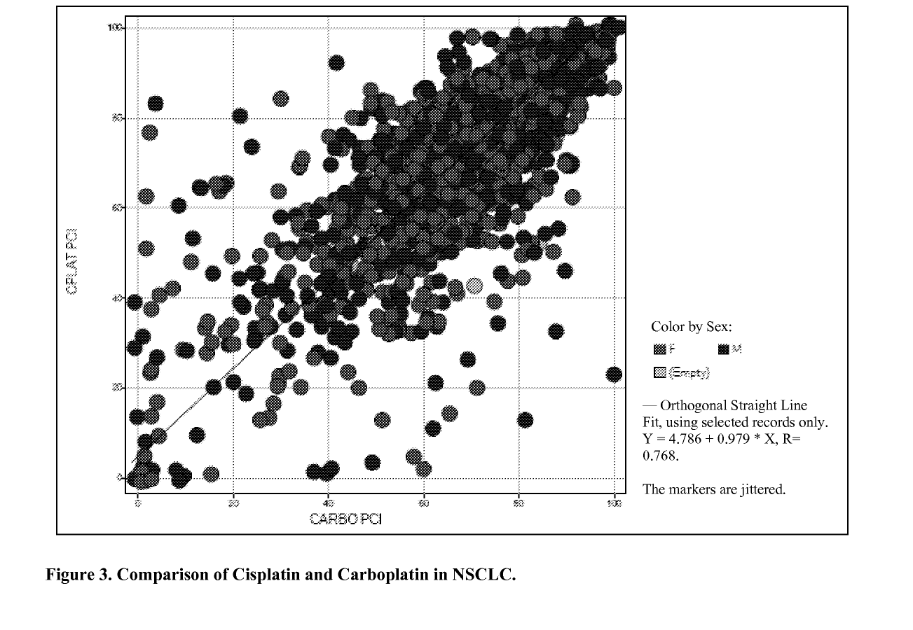

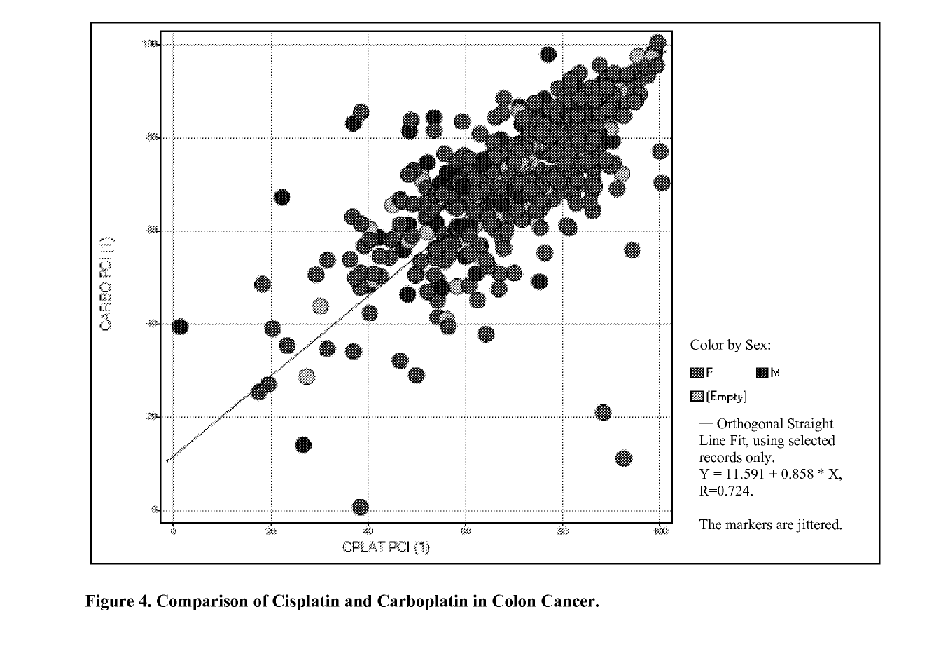

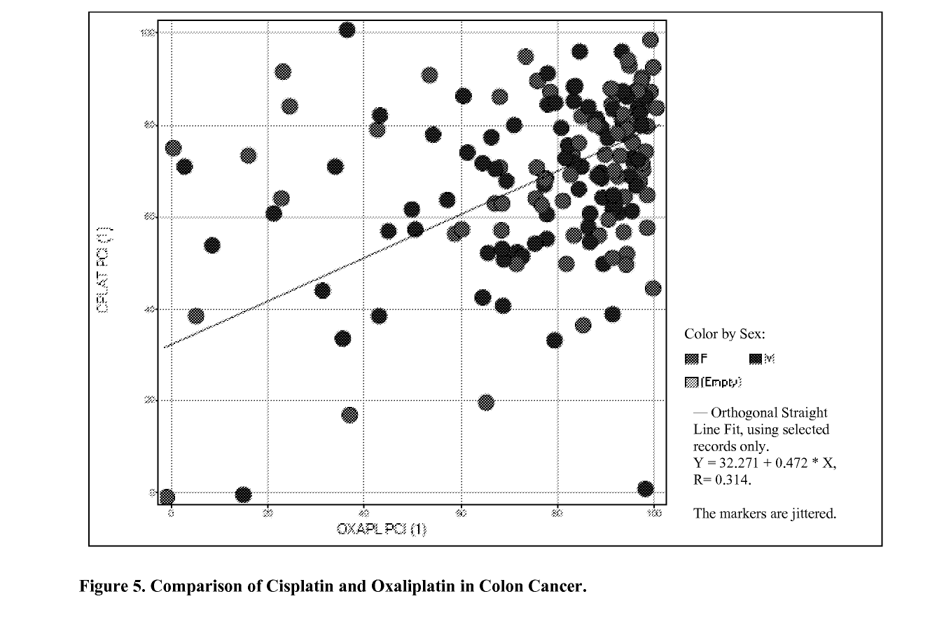

Results of the comparison of Cisplatin and Carboplatin resistance or sensitivity in overian cancer and NSCLC samples are shown in FIGS. 2-4. The results in FIG. 2 demonstrated the similarity of the response of ovarian cancer tumors to both Cisplatin and Carboplatin. Due to the high correlation (r=0.749), amplicons for Cisplatin resistance most likely also are useful for predicting Carboplatin resistance. The results shown in FIGS. 3-4 demonstrated that resistance to Carboplatin was also very similar in NSCLC (r=0.768) (FIG. 3) and colon cancer (r=0.724) (FIG. 4). However, the results in FIG. 5 demonstrated that the correlation of the response of colon cancer tumors to both Cisplatin and Oxaliplatin is not as strong as that observed between Cisplatin and Carboplatin (r=0.14). Therefore, differences in the amplicons for predicting Oxaliplatin response may be expected to vary when compared to those discovered in Cisplatin and Carboplatin resistant tumors.

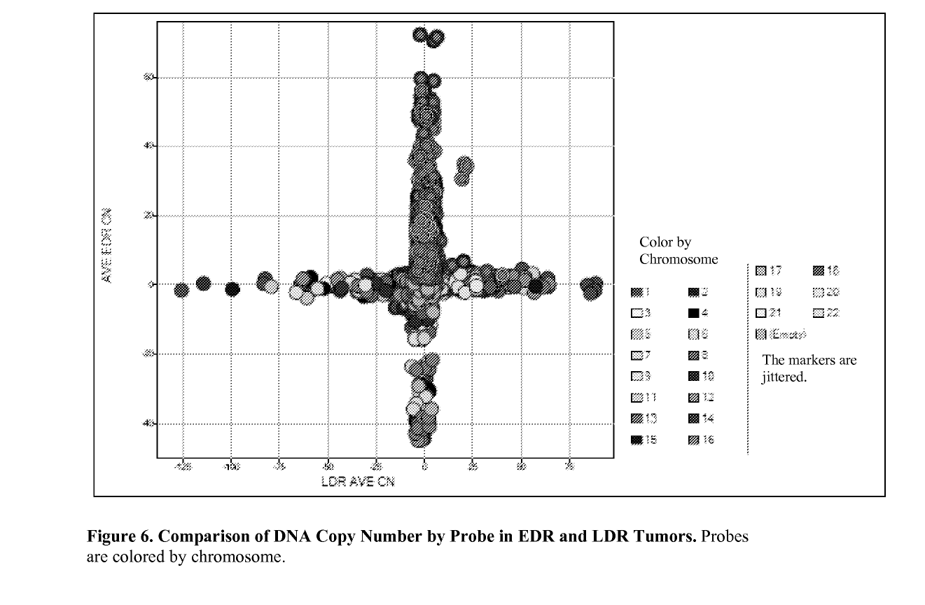

The GSA Copy Numbers were determined by the Affymetrix Chromosome Copy Number Tool for these tumors and were divided by the GSA Copy Number for the NHDFs. These data were then imported into DecisionSite Version 8.2.1 (Spotfire Inc, Boston Mass.). The adjusted DNA Copy Numbers were plotted as Average EDR (Y axis) versus Average LDR (X axis) as seen in FIG. 6. The probes that had at least a four-fold increase in DNA copy number as compared to the Average LDR DNA Copy Number were selected. A further limitation was placed on the probes from the Average EDR group by having at least three of the eight probes show a greater than a five-fold increase. This was done to account for a single outlier that could raise the average while only one tumor had a significant increase in DNA.

Table 1 shows the DNA regions found to be altered in Cisplatin resistant ovarian cancer. The number of probes listed in the table indicates the number of probes within that cytoband that demonstrated an increase in copy number.

Similarly, Table 2 details the DNA regions found to be altered in Oxaliplatin-resistant colon cancer. Again, the number of probes listed in the table indicates the number of probes within that cytoband that demonstrated an increase in copy number.

Finally, Table 3 demonstrates the regions of DNA amplification that occur in both tumor types to both Cisplatin and Oxaliplatin, i.e., regions of DNA that are amplified in both Cisplatin EDR ovarian tumors and Oxaliplatin-resistant colon tumors.

It should be understood that the foregoing disclosure emphasizes certain specific embodiments of the invention and that all modifications or alternatives equivalent thereto are within the spirit and scope of the invention as set forth in the appended claims.

TABLE 1

| Chromosome | Cytoband | Number of Probes |
|---|---|---|
| 1 | q41 | 25 |
| 2 | p12 | 23 |
| 3 | p24 | 11 |
| 3 | q13 | 5 |
| 3 | q25 | 11 |
| 3 | q26 | 6 |
| 4 | q21 | 18 |
| 4 | q32 | 37 |
| 5 | q33 | 13 |
| 7 | q32 | 10 |
| 7 | q34 | 6 |
| 8 | p23 | 43 |
| 8 | q13 | 5 |
| 8 | q24 | 18 |
| 9 | q31 | 7 |
| 10 | p14 | 13 |
| 11 | p13 | 9 |
| 11 | p15 | 7 |
| 12 | q21 | 11 |
| 12 | q22 | 10 |
| 12 | q23 | 9 |
| 13 | q13 | 22 |
| 13 | q33 | 24 |
| 15 | q25 | 6 |
| 16 | p13 | 11 |
| 16 | q23 | 5 |
| 17 | p13 | 8 |
| 19 | p12 | 6 |

TABLE 2

| Chromosome | Cytoband | Number of Probes |
|---|---|---|
| 1 | p31 | 22 |
| 1 | q24 | 19 |
| 1 | q41 | 17 |
| 2 | q22 | 27 |
| 3 | p12 | 25 |
| 3 | p14 | 36 |
| 3 | p26 | 55 |
| 3 | q13 | 18 |
| 3 | q22 | 19 |
| 3 | q26 | 15 |
| 4 | p15 | 24 |
| 4 | q13 | 26 |
| 5 | p13 | 28 |
| 5 | q15 | 19 |
| 5 | q23 | 28 |
| 6 | p12 | 69 |
| 6 | q16 | 17 |
| 6 | q21 | 28 |
| 6 | q24 | 26 |
| 7 | p21 | 94 |
| 7 | q35 | 28 |
| 8 | p22 | 25 |
| 8 | p23 | 19 |
| 8 | q13 | 17 |
| 8 | q23 | 31 |
| 8 | q24 | 19 |
| 9 | p23 | 41 |
| 9 | q33 | 20 |
| 11 | p14 | 31 |
| 11 | q14 | 39 |
| 12 | q21 | 21 |
| 13 | q31 | 36 |
| 13 | q33 | 20 |
| 14 | q22 | 18 |
| 14 | q31 | 64 |
| 16 | q23 | 15 |
| 18 | q12 | 15 |
| 18 | q22 | 65 |
| 20 | p12 | 15 |
| 21 | q21 | 39 |

TABLE 3

| Chromosome | Cytoband |
|---|---|
| 1 | q41 |
| 3 | q13 |
| 3 | q26 |
| 8 | p23 |
| 8 | q13 |
| 13 | q33 |
| 16 | q23 |

We claim:

1. A method for identifying a human tumor that is resistant to a chemotherapeutic drug, comprising the step of assaying one or a plurality of genetic loci from a chromosomal region in the tumor, wherein the tumor is resistant to the chemotherapeutic drug if at least one of the genetic loci is present in differential amounts in the tumor.

2. The method of claim 1, wherein the tumor is resistant to the chemotherapeutic drug if at least one of the genetic loci is amplified in the tumor.

3. The method of claim 1, wherein the tumor is resistant to the chemotherapeutic drug if at least one of the genetic loci is deleted in the tumor.

4. The method of claim 1 wherein the tumor is a breast cancer tumor, colon cancer, ovarian cancer tumor, or non-small cell lung cancer tumor.

5. The method of claim 1 wherein the drug is a platinum-based chemotherapeutic drug.

6. The method of claim 5 wherein the drug is Cisplatin, Carboplatin, or Oxaliplatin.

7. The method of claim 1 wherein the chromosomal region amplified is located at chromosome 1q41, 3q13, 3q26, 8p23, 8q13, 13q33, or 16q23.

8. The method of claim 7 that is used to predict resistance to Cisplatin, Carboplatin, or Oxaliplatin.

9. The method of claim 1 wherein the chromosomal region amplified is located at chromosome 1q41, 2p12, 3p24, 3q13, 3q25, 3q26, 4q21, 4q32, 5q33, 7q32, 8p23, 8q13, 8q24, 9q31, 10p14, 11p13, 11p15, 12q21, 12q22, 12q23, 13q13, 13q33, 15q25, 16p13, 16q23, 17p13, or 19p12.

10. The method of claim 9 that is used to predict resistance to Cisplatin and Carboplatin.

11. The method of claim 1 wherein the chromosomal region amplified is located at chromosome 1p31, 1q24, 1q41, 2q22, 3p12, 3p26, 3q13, 3q22, 3q26, 4p15, 4q13, 5p13, 5q15, 5q23, 6p12, 6q16, 6q21, 6q24, 7p21, 7q35, 8p22, 8p23, 8q13, 8q23, 8q24, 9p23, 9q33, 1p14, 11q14, 12q21, 13q31, 13q33, 14q22, 14q31, 16q23, 18q12, 18q22, 20p12, or 21q21.

12. The method of claim 11 that is used to predict resistance to Oxaliplatin.

13. The method of claim 1, wherein the tumor cells are separated from the tumor sample.

14. The method of claim 1, wherein amplification of one or a plurality of genes or genetic loci from a chromosomal region are detected by gene arrays, fluorescence in situ hybridization (FISH), Southern blot, polymerase chain reaction (PCR), enzyme-linked immunosorbent assay (ELISA) or comparative genomic hybridization (CGH).

15. The method of claim 1, wherein overexpression of one or a plurality of genes or genetic loci are detected by assaying RNA expression.

16. The method of claim 15, wherein RNA overexpression is detected by gene expression arrays, RT-PCR, or Northern blots.

17. The method of claim 1, wherein overexpression of one or a plurality of genes is detected by assaying protein expression.

18. The method of claim 17, wherein protein overexpression is detected by western blot, ELISA, immunohistochemistry or mass spectroscopy.

19. A method of claim 1, wherein said genetic locus encodes a microRNA.

20. A method of claim 19, wherein differential amounts of a microRNA-encoding genetic locus is detected by array, polymerase chain reaction (PCR), or in situ hybridization (ISH).

21. A kit for assaying differential amounts of one or a plurality of genes or genetic loci, comprising at least one probe specific for any said genes or genetic loci.

22. The kit of claim 21, wherein the kit is for assaying amplification or overexpression of one or a plurality of genes or genetic loci.

23. The kit of claim 21, wherein the kit is for assaying deletion of one or a plurality of genes or genetic loci.

24. The kit of claim 21 wherein the amplified genes or genetic loci are located at chromosome 1q41, 3q13, 3q26, 8p23, 8q13, 13q33, and 16q23 that is used to predict resistance to Cisplatin, Carboplatin, or Oxaliplatin.

25. The kit of claim 21 wherein the chromosomal region amplified is located at chromosome 1q41, 2p12, 3p24, 3q13, 3q25, 3q26, 4q21, 4q32, 5q33, 7q32, 8p23, 8q13, 8q24, 9q31, 10p14, 11p13, 11p15, 12q21, 12q22, 12q23, 13q13, 13q33, 15q25, 16p13, 16q23, 17p13, or 19p12 that is used to predict resistance to Cisplatin or Carboplatin.

26. The kit of claim 21 wherein the chromosomal region amplified is located at chromosome 1p31, 1q24, 1q41, 2q22, 3p12, 3p26, 3q13, 3q22, 3q26, 4p15, 4q13, 5p13, 5q15, 5q23, 6p12, 6q16, 6q21, 6q24, 7p21, 7q35, 8p22, 8p23, 8q13, 8q23, 8q24, 9p23, 9q33, 1p14, 11q14, 12q21, 13q31, 13q33, 14q22, 14q31, 16q23, 18q12, 18q22, 20p12, or 21q21 that is used to predict resistance to Oxaliplatin.

27. The kit of claim 21, wherein the genes are within those genetic regions are evaluated for amplification by gene arrays, PCR, RT-PCR, or in situ hybridization.

28. The kit according to claim 21, further comprising a detectable label for labeling each of said probes.

29. The kit of claim 21, wherein the probes are detectably labeled.

30. One or a plurality of probes for a plurality of genes or genetic loci that are differentially expressed in a tumor that is resistant to a chemotherapeutic drug, wherein each probe specifically binds or hybridizes to a gene or genetic locus located within 1q41, 3q13, 3q26, 8p23, 8q13, 13q33, or 16q23, chromosome 1q41, 2p12, 3p24, 3q13, 3q25, 3q26, 4q21, 4q32, 5q33, 7q32, 8p23, 8q13, 8q24, 9q31, 10p14, 11p13, 11p15, 12q21, 12q22, 12q23, 13q13, 13q33, 15q25, 16p13, 16q23, 17p13, or 19p12, or chromosome 1p31, 1q24, 1q41, 2q22, 3p12, 3p26, 3q13, 3q22, 3q26, 4p15, 4q13, 5p13, 5q15, 5q23, 6p12, 6q16, 6q21, 6q24, 7p21, 7q35, 8p22, 8p23, 8q13, 8q23, 8q24, 9p23, 9q33, 11p14, 11q14, 12q21, 13q31, 13q33, 14q22, 14q31, 16q23, 18q12, 18q22, 20p12, or 21q21.
